(19) United States
(12) Patent Application Publication
(10) Pub. No.: US 2026/0306147 A1
(43) Pub. Date: Oct. 8, 2026

(54) IMPACTED BONE ANCHOR AND METHODS

(71) Applicants: Jason Blain, Encinitas, CA (US); Greg Martin, Carlsbad, CA (US)

(72) Inventors: Jason Blain, Encinitas, CA (US); Greg Martin, Carlsbad, CA (US)

(21) Appl. No.: 19/098,737

(22) Filed: Apr. 2, 2025

(51) Int. Cl. A61B 17/80; A61B 17/86

(52) U.S. Cl. CPC A61B 17/80; A61B 17/86; A61B 2017/8655

(57) ABSTRACT

A bone anchor comprising an enlarged head and an elongate bone screw body having a shape conducive for linear impacted introduction into bone. Helical flanges extend about the body of the bone anchor and are interrupted by a plurality of elongate axial grooves. The helical flanges can be aligned or misaligned. The bone anchors can be either cannulated bone anchors or solid anchors absent of cannulation. The bone anchors can be utilized in a variety of applications such as to secure an intervertebral spacer, a bone plate, or for fixation across bone segments such as when inducing fusion at an SI joint. The bone anchor can include an elongate window for the introduction of bone material to enhance fusion. In one method, the bone anchor is impacted into the bone and then rotated to continue advancement of the bone anchor into the bone.

This application is a Non-Provisional Patent Application.

### BACKGROUND OF THE INVENTION

Field of the Invention. The invention relates generally to bone anchors utilized in orthopedic surgery, and more particularly to bone anchors that are inserted by impacting the bone anchor into bone.

Description of Related Art. Prior devices consisted of bone anchors resembling screws with a helix thread for threaded attachment into bone. Insertion of these screws into bone consists of driving the screw in rotation along its long axis. Some bone anchors of the prior art could be impacted into bone, such as pins or triangular dowels, however, these devices have limited sustained fixation.

### SUMMARY OF THE INVENTION

Disclosed herein are bone anchors for fixation of bone. The bone anchors can be utilized for the fixation of multiple bones or bone fragments, or for fixation of other orthopedic implants to bone such as for example, an intervertebral disc or bone plate.

In one form, a bone anchor is initially impacted into bone along a linear axis then rotated after impaction resulting in consequent compression and greater fixation hold from back out.

In one form, a bone anchor has a pointed leading tip to reduce the initial insertion force into bone.

In one form, the bone anchor has a shape profile conducive for impaction into bone wherein upon insertion the stresses on bone are managed to avoid undesired bone fracture.

In one form, the bone anchor comprises helical flanges (bone screw threads) encircling its perimeter wherein the helical flanges are interrupted by axial grooves whereby rotating the bone anchor after impaction into bone causes the threads to cut into the bone previously undisturbed by the impaction. This results in consequent compression between the head of the bone anchor and the bone.

In one form, the helical flanges are aligned along a helix.

In one form, the helical flanges are misaligned.

In one form, the bone anchor comprises a plurality of longitudinally placed axial grooves extending from the distal tip of the bone anchor to the neck of the bone anchor. The axial grooves interrupt the helical flanges (bone screw threads) at various angles around the central axis of the bone anchor.

In one form, the axial grooves are parallel to the central axis of the bone anchor.

In one form, the axial grooves diverge toward the central axis as they approach the distal tip of the bone anchor.

In one form, the trailing geometry of the bone anchor has a drive head for driving the bone anchor into bone.

In one form, the drive head comprises a profile for receiving a driver instrument such as for example, a hex, torx, or other geometry capable of imparting torsion on the bone anchor.

In one form, consequent to torque being applied by a driver to the drive head of the bone anchor, the helical flange segments of the device engage the bone with consequent compression.

In one form, the bone anchor comprises a counter-rotation body that resists bone anchor rotation in a direction opposite the insertion torque.

In one form, the counter-rotation body comprises a counter-rotation lead in the form of a step or a barb.

In one form, the bone anchor is initially impacted into bone utilizing the axial grooves to reduce bone stress upon entry into the bone.

In one form, a pilot hole is drilled in the bone to guide the bone anchor to a predetermined location. The pre-drilled hole can reduce the impact force required to seat the bone anchor into its desired position while also reducing bone stress.

In one form, a guide wire can be seated in a bone and wherein a bone anchor is cannulated and can follow the path of the prepositioned guidewire.

In one form, following impaction, the bone anchor may be rotated with a driver instrument (anchor driver) that engages a drive recess at the trailing end of the device.

In one form, the bone anchor is utilized for fixating an intervertebral body implant to vertebrae of the spine.

In one form, the bone anchor is utilized to fixate and compress bone fractures.

In one form, the bone anchor is utilized to fixate a bone plate to bone.

In one form, the bone anchor is used to fixate and compress a sacroiliac joint.

In one form, helical flanges of the bone anchor have an interrupted thread pattern.

In one form, the thread pattern is interrupted by axial grooves.

In one form, the bone anchor reduces time required to implant orthopedic fixation devices.

In one form, the bone anchor has a generally spherical head.

In one form, the bone anchor has at least a partially conical head.

In one form, the conical portion functions to compress a bone fracture when the anchor is used stand-alone for fraction fixation.

In one form, the major diameter of the bone anchor enlarges as it approaches the neck of the bone anchor.

In one form, the major diameter of the bone anchor is substantially constant.

In one form, the bone anchor engages previously undisturbed bone upon rotation of the bone anchor leading to better mechanical fixation.

### BRIEF DESCRIPTION OF THE SEVERAL VIEWS OF THE DRAWINGS

These and other features and advantages of the present invention will become more readily appreciated when considered in connection with the following detailed description and appended drawings, wherein each drawing is according to one or more embodiments shown and described herein, and wherein:

FIG. 1 depicts a perspective view of a bone anchor;

FIG. 2 depicts a side view of the bone anchor of FIG. 1;

FIG. 2B depicts a distal end view of the bone anchor of FIG. 1 showing axial grooves converging toward the distal end;

FIG. 2C is a cross-sectional view of the bone anchor of FIG. 1;

FIG. 3 is a partial close-up perspective view of a counter-rotation feature of the bone anchor depicted in FIG. 1;

FIG. 4 is a proximal end partial perspective view of the bone anchor of FIG. 1;

FIG. 5 is a side view of a bone anchor with drive recesses removed;

FIG. 5B is a side view of an alternative bone anchor with drive recesses removed;

FIG. 5C is a side view of an alternative bone anchor with drive recesses removed;

FIG. 5D is a side view of an alternative bone anchor with drive recesses removed;

FIG. 5E is a side view of an alternative bone anchor with drive recesses removed;

FIG. 6 is an inferior perspective view of an orthopedic fixation device comprising the bone anchors of FIG. 1 utilized within an intervertebral spacer;

FIG. 7 is a lateral view of an orthopedic fixation device comprising the bone anchors of FIG. 1 utilized within an intervertebral spacer;

FIG. 8 is a superior perspective view of the intervertebral spacer of FIG. 7;

FIG. 9 is a superior perspective view of the intervertebral spacer of FIG. 6;

FIG. 10 is an anterior perspective view of the intervertebral spacer of FIG. 6 implanted between opposing vertebrae of a human spine;

FIG. 11 is a perspective view of a blocking cam;

FIG. 12 is a perspective view of an opposing side of the blocking cam of FIG. 11;

FIG. 13 is a perspective view of an opposing side of the blocking cam of FIG. 11;

FIG. 14 is a perspective view of an opposing side of the blocking cam of FIG. 11;

FIG. 15 is a perspective view of a bone anchor as might be used in a bone plate;

FIG. 16 is a head perspective view of the bone anchor of FIG. 15;

FIG. 17 is a perspective view of an orthopedic fixation device in the form of a bone plate utilizing the bone anchors of FIG. 16 (two anchors removed);

FIG. 18 is an opposed perspective view of the orthopedic fixation device of FIG. 17;

FIG. 19 is a perspective view of the orthopedic fixation device of FIG. 17 implanted against a human spine;

FIG. 20 is a distal perspective view of a cannulated bone anchor;

FIG. 21 is a proximal perspective view of a non-cannulated bone anchor;

FIG. 22 is a cross-sectional view of the bone anchor of FIG. 20;

FIG. 23 is a lateral view of the cannulated bone anchor of FIG. 20;

FIG. 24 is a cross-sectional view of the non-cannulated bone anchor of FIG. 21;

FIG. 25 is a lateral view of the non-cannulated bone anchor of FIG. 21;

FIG. 26 is a lateral view of an anchor driver implanting a sacral bone anchor in a pelvis with an additional view of an anchor driver with anchor attached;

FIG. 27 is a cross-sectional view of anchor drivers implanting sacral bone anchors in a pelvis;

FIG. 28 is a flow diagram depicting a method to secure a bone plate using bone anchors;

FIG. 29 is a flow diagram depicting a method to secure an intervertebral spacer using bone anchors;

FIG. 30 is a flow diagram depicting a method to secure an SI joint using a bone anchor;

FIG. 31 is a flow diagram depicting a method of introducing bone fusion material into a bone anchor and compressing it into elongate windows for fusion with surrounding bone.

### DETAILED DESCRIPTION OF SELECTED EMBODIMENTS OF THE INVENTION

Select embodiments of the invention will now be described with reference to the Figures. Like numerals indicate like or corresponding elements throughout the several views and wherein various embodiments are separated by letters (i.e. 100, 100B, 100C). The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

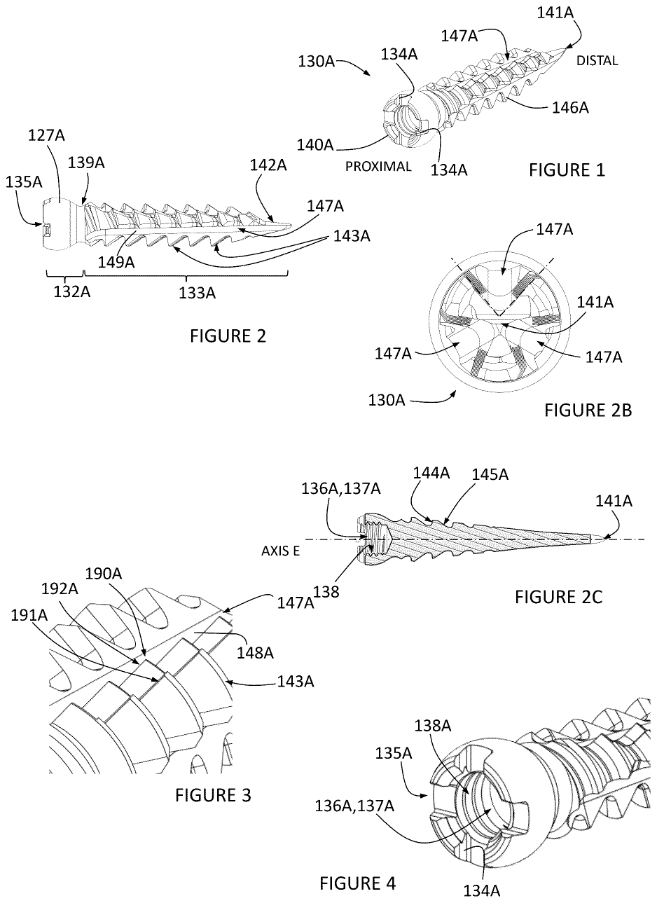

FIGS. 1-5 depict views of one type of bone anchor that can be used within a orthopedic fixation device 100A. The bone anchors 130A in this embodiment are similar to traditional bone screws and comprise an elongate screw body 133A that further comprises bone screw threads 143A along the length of the screw body 133A of the bone anchor. The bone screw threads are interrupted by a plurality of elongate axial grooves 147A, defined by groove face 148A, partially or fully inset into the threads and which extend from distal tip 141A towards neck 139A. In this embodiment, the axes of individual axial grooves 147A are coplanar with the long axis of the screw (i.e. axis E) while simultaneously converging toward axis E to follow the changing minor diameter which tends to be smaller distally and larger proximally. This is depicted in FIG. 2B where the axial grooves 147A splay outward as they approach enlarged head 132A. The depth of the axial grooves 147A can vary between being shallower than the minor diameter of the anchor, to being equal, to being deeper than the minor diameter of the anchor. In preferred embodiments, and also as noted in FIG. 2B by the broken lines, the cross-sectional profile of the axial grooves is a substantially rounded V-shape, however other shape profiles can be utilized. Again, note in this bone anchor embodiment, the minor diameter of the anchor increases from tip to head. In this embodiment, the bone anchor 130A is first impacted along its central axis E to seat in the bone with distal tip 141A entering the bone first. Bone anchor 130A can then be advanced by rotation along its central axis E for final fixation into bone such as into a vertebral body for example. The bone anchors can be said to have bone screw threads that are interrupted by axial grooves. However, as an alternative, the bone screw threads can also be described as helical flanges especially in embodiments where the helical flanges are not aligned across an axial groove.

As depicted in FIG. 1, the proximal end of the bone anchor 130A comprises an enlarged head 132A which is at least partially spherical as defined by a head surface 127A. A drive recess 135A extends through proximal face 140A into the enlarged head from a proximal end. The drive recess 135A is defined by a plurality of drive surfaces 134A configured for engagement by a drive tool that can impart rotation on the bone anchor 130A along axis E. Advancing deeper past drive recess 135A, is head lock 136A which is defined by head threads 138A. This feature can be utilized during surgery for example, for securing the bone anchor to a threaded end of an inserter tool. A narrowed neck 139A joins enlarged head 132A with screw body 133A. At the distal end of bone anchor 130A is distal tip 141A which can be sharpened and in some cases a tip lead 142A is present that leads into the interrupted bone screw threads. In the event of a threaded bone anchor, the bone screw threads 143A comprise a proximal thread face 144A facing substantially proximal, and a distal thread face 145A facing substantially distally. An edge face 146A joins the distal thread face 145A and proximal thread face 144A and merges into rounded V-shaped groove face 149A that defines axial groove 147A. As noted in FIG. 2C, the proximal thread face can be substantially perpendicular to the elongate axis (axis E) of the bone anchor, whereas the distal thread face can be sloped thereby providing the screw more resistance to pullout yet eased insertion.

As depicted in FIG. 3, some embodiments of the bone anchors comprise a counter-rotation body 190A that resists bone anchor rotation in a direction opposite the insertion torque. The counter-rotation body comprises a counter-rotation lead 192A in the form of a counter-rotation step 191A or barb.

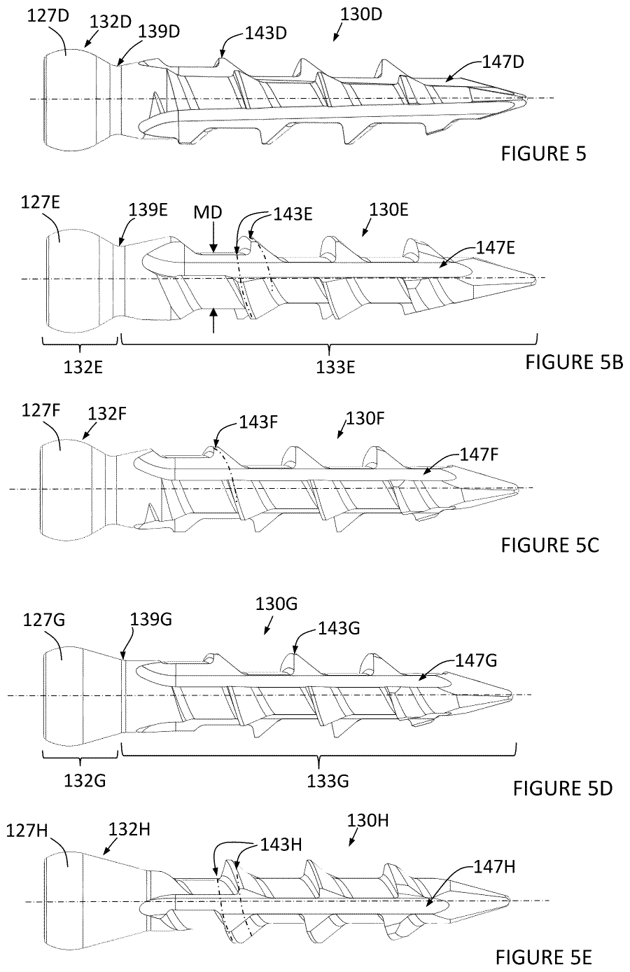

FIGS. 5 through 5E depict alternative embodiments of bone anchors having varying features. Features depicted in any one of the embodiments disclosed herein can be utilized to form other variations of the invention. The bone anchor depicted in FIG. 5 is much like the bone anchor depicted previously in FIG. 2. It comprises a generally spherical enlarged head 132D, bone screw threads 143D (helical flanges) aligned across the axial grooves, and a tapered axial groove 147D and minor diameter (MD). The bone anchor of FIG. 5B also has a spherical shaped enlarged head 132E, but with bone screw threads 143E that are mis-aligned across axial groove 147E as depicted by the dotted lines, and wherein the axial groove and minor diameter (MD) is generally parallel to the central axis of the bone anchor 130E. The bone anchor 130F of FIG. 5C also has a spherical shaped enlarged head 132F with aligned bone screw threads 143F, and an axial groove and minor diameter that is generally parallel to the central axis of the bone anchor. The bone anchor of FIG. 5D has an enlarged head 132G that is conically shaped on the distal side of the head. The bone screw threads 143G are aligned on either side of the axial grooves 147G, and the axial grooves and minor diameter are generally parallel to the central axis of the bone anchor. The bone anchor of FIG. 5E again has a conical shaped enlarged head 132H on the distal side of the head. The bone screw threads 143H are again aligned, and it has axial grooves 147H and a minor diameter that is generally parallel to the central axis of the bone anchor. The drive profiles in the enlarged head of these embodiments (FIG. 5-5E) mirror that depicted in FIG. 16. Note that the bone anchors of FIG. 5-5D have a minor diameter that enlarges on the anchor as it approaches the neck of the screw. This feature gives the screw additional strength in this region such as when it used to secure a bone plate. In some cases, this enlarged minor diameter area angles downward towards the neck to allow pivoting motion of the anchor. The conical enlarged head feature assists in compressing a bone fracture when the bone anchor is used for stand-alone fraction fixation.

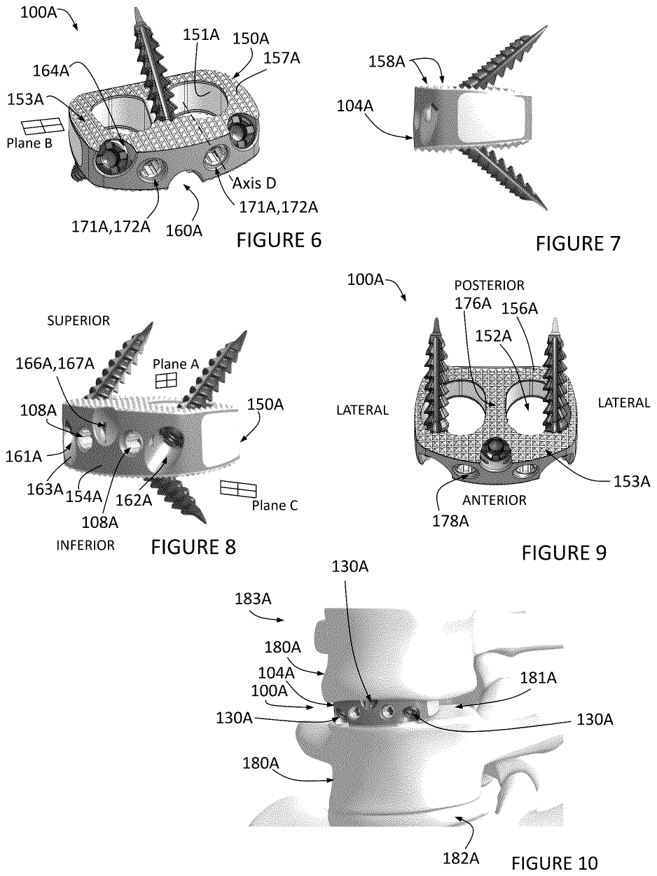

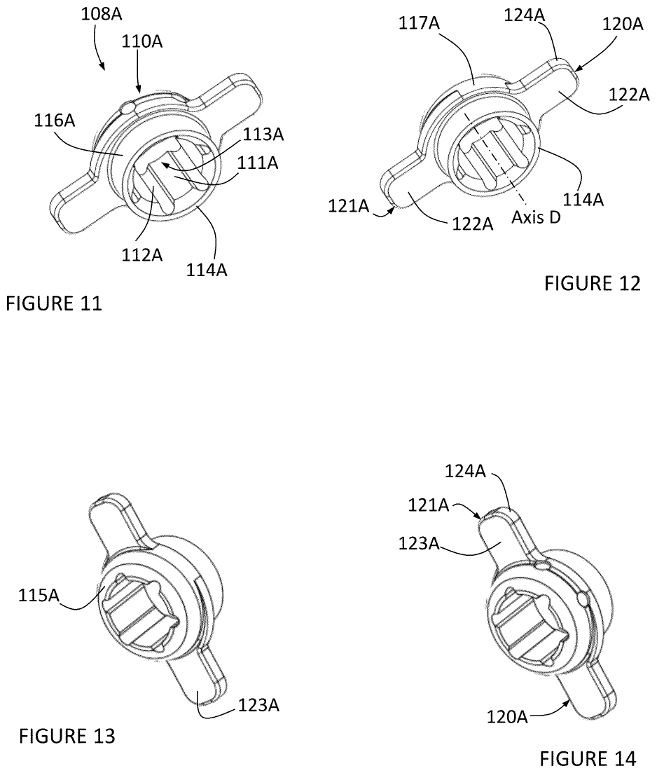

When used for implanting an orthopedic fixation device, the bone anchors are advanced through respective fixator ports of a spacer body or bone plate and into the bone of a recipient. After fully advancing the bone anchor, an anti-back out device integrated into the intervertebral spacer or bone plate can be used to prevent unintended back out of the bone anchor. For example, a blocking device that can be used is in the form of a blocking cam 108A as depicted in FIGS. 11-14. FIGS. 6-10 depict the blocking cam utilized within an intervertebral spacer 104A. With the blocking cam in an unlocked mode and consequently not obstructing the respective fixator port (i.e. central fixator port 160A, 1^{st} lateral fixator port 161A, 2^{nd} lateral fixator port 162A), the bone anchor can be inserted or removed. Once fully inserted in the fixator port, the blocking cam is then rotated to a locked mode such that the cam tabs (first tab 120A, second tab 121A) on the blocking cam 108A interfere with the respective fixator port thereby blocking removal of the bone anchor. The locked mode prevents the bone anchor from backing out of the fixator ports thereby preventing or limiting undesired bone anchor disassociation or backout.

Described here and depicted in FIG. 6-10 are select embodiments of an implantable orthopedic fixation device for supporting bony structures. As previously noted, the orthopedic fixation devices can comprise one or more fixator ports for seating a bone anchor therein and can further comprise an integrated blocking cam that blocks the port passageway when activated to a locked mode thereby preventing back out of the bone anchor once the bone anchor is advanced and seated in adjacent bone. The orthopedic fixation device can be in the form of a fixation plate, otherwise known as a bone plate 200B (FIG. 17-19), or as illustrated in FIGS. 6-10, is in the form of an intervertebral spacer 104A operable for fixation in the intervertebral space 181A between two vertebral bodies 180A when at least a portion of an intervertebral disc 182A is removed. FIG. 10 depicts the orthopedic fixation device 100A in an implanted mode wherein bone anchors 130A extend through the spacer body 150A into adjacent vertebrae.

A wide variety of intervertebral spacers known in the art can be adapted to use the disclosed impacted bone anchor. In the intervertebral spacer embodiment of FIGS. 6-10, intervertebral spacer 104A is defined by a boundary wall 153A defining the outside perimeter of the spacer. The boundary wall is bounded on a superior end by a superior face 156A, inferiorly by an inferior face 157A, and radially by an external face 154A. The intervertebral spacer 104A can also include a dividing wall 176A also extending between the inferior face and superior face and merging with the boundary wall 153A. An internal face 151A, spaced inward from external face 154A on the boundary wall and on the dividing wall, defines one or more bone cavities 152A for housing bone or bone substitute during the fusion process. Dividing wall 176A is positioned to provide additional support between the vertebral end plate above and the vertebral end plate below and consequently divides the bone cavities into additional bone cavities. As noted in the Figures, the superior face 156A and inferior faces 157A are preferably substantially convex to match the contour of typical vertebral endplates. In preferred embodiments, extending from the inferior face and superior face are a plurality of bone teeth 158A. The bone teeth are typically pointed and/or ridged to engage the adjacent end plates and thereby minimize movement therebetween the implant and endplate.

Intervertebral spacer 104A comprises a first lateral fixator port 161A offset laterally on one side of the intervertebral spacer, a second lateral fixator port 162A offset laterally on the opposite side, and a central fixator port 160A generally centered on the spacer. The first and second lateral fixator ports are directed from the external face 154A upwards (superiorly) through the bone facing superior face 156A, whereas the central fixator port is directed downwards through the bone facing inferior face 157A. It is recognized that these directions can be reversed in alternative embodiments. Each fixator port is configured to house a bone anchor for engaging the adjacent intervertebral endplate when implanted. The fixator ports are substantially cylindrical in this embodiment however can assume other profiles. The first part of the fixator ports are defined by tunnel face 163A which extends from external face 154A having a diameter sufficient to pass the enlarged head 132A of the bone anchor 130A. The fixator port then narrows as defined by neck face 164A that is sufficiently small in diameter to offer a seat for enlarged head 132A yet is sufficiently large in diameter to pass screw body 133A of bone anchor 130A. The final section of the fixator port is defined by shank face 165A which can be sufficient in diameter to pass the screw body 133A of bone anchor 130A while simultaneously limiting off axis movement or have more substantial diameter to provide some level of off axis movement. As noted in the Figures, a portion of the fixator ports can open to the bone cavities 152A as well as to superior face 156A and inferior face 157A. The blocking cams 108A are housed along a common axis D within cam ports 172A (FIG. 6-10) that are positioned in boundary wall 153A of spacer body 150A.

A tab guide face 166A extends from the cam port 172A to adjacent lateral fixator ports 161A, 162A to define cam windows 167A in which the respective first tab 120A or second tab 121A rotate and extend through to create a retractable barrier in a respective fixator port. In this case, cam window 167A has a substantially rectangular profile but can vary as needed to complement the tabs.

The intervertebral spacer can assume a wide variety of profiles. For example, as depicted in FIG. 1, external face 154A defines a profile that is substantially lima bean shaped or a smaller version of a human endplate. However, the intervertebral spacer can assume other profiles as noted in the prior art such as generally rectangular. As further depicted in the Figures, one of the bone anchors is downward directed and sits midline, whereas two of the bone anchors are upward directed and spaced laterally from the downward directed anchor. This arrangement can be varied without loss of function in alternative embodiments.

The orthopedic fixation device 100A comprises an integrated blocking mechanism 106A for preventing undesired back out of the bone anchors once implanted. In the embodiments depicted, the blocking mechanism is in the form of a blocking cam 108A. The blocking cam 108A (FIG. 11-14) comprises a round central body 110A with an internal central face 111A spaced about axis D defining a central drive pocket 113A extending through the central body from an outward facing outward face 114A to an inward facing inward face 115A. A plurality of drive faces 112A extend into the central faces for engagement with a camming tool that can impart a rotary force on the blocking cam to switch between a locked mode (where first tab 120A or second tab 121A block removal of the bone anchor) and an unlocked mode. An outer taper 125A can be present between the outward face 114A and central face 111A to ease insertion of a camming tool. Stepped out from outward face 114A, a pivot face 116A defines a generally cylindrical outer portion of central body 110A. Adjacent pivot face 116A is head face 117A which defines a generally cylindrical outer portion of central body 110A having an enlarged diameter compared to pivot face 116A. Distally, head face 117A rounds down until terminating at inward face 115A. The rounding of head face 117A is configured in size and rounded shape to seat within pivot seat 171A of the cam port 172 for pivoting therebetween when switching between locked and unlocked modes.

Laterally extending from head face 117A of central body 110A of the blocking cam 108A is a pair of opposed tabs (first tab 120A, second tab 121A. Again, the blocking cam can switch from an unlocked mode to a locked mode when the central body is rotated. In the locked mode, the tabs extend into and interfere with the bone-engagement passageway (fixator ports) and prevent component disassociation such as bone anchor backout. Proximally, first tab 120A and second tab 121A are defined by a substantially flat tab outer face 122A facing outward and by an opposed substantially flat tab inner face 123A facing inward towards the intervertebral spacer. A tab radial face 124A, extends between the tab inner face and tab outer face.

The first tab 120A and second tabs 121A extend through cam windows 167A into fixator ports in a locked mode. The first tab 120A and second tabs 121A are retracted into cam windows 167A in an unlocked mode thereby providing full access for insertion and removal of bone anchors 130A through the fixator ports.

Depicted in FIG. 10, the intervertebral spacer 104A of the orthopedic fixation device 100A is inserted into an intervertebral space 181A between adjacent vertebral bodies 180A of a human spine 183A. As shown, the intervertebral spacer 104A of the orthopedic fixation device is positioned on the surface of bone structures which in this case are between the endplates of adjacent vertebrae.

During implantation, the bone anchors are advanced through respective fixator ports and impacted into the bone of a recipient. The bone anchors can then be rotated by a driver to engage bone uncorrupted during impaction. Rotation may vary, however, in general, a bone anchor with two opposed axial grooves would be turned 90 degrees, whereas a bone anchor with three equally spaced axial grooves would be rotated 60 degrees, and a bone anchor with four equally spaced axial grooves would be rotated 45 degrees. In alternative embodiments, the bone anchor can be advanced with axial force an amount greater than just described to threadably continue to advance the anchor. However, the bone anchor offers the best resistance to backout when the threads of the anchor are misaligned with the bone channels created in the bone when the bone anchor is initially impacted.

After advancing the bone anchor with the blocking cam in an unlocked mode (and consequently unobstructing the respective fixator port), the blocking cam is then rotated to a locked mode such that the cam tabs (first tab 120A, second tab 121A) on the blocking cam 108A interfere with the respective fixator port. The locked mode prevents the bone anchor from backing out of the fixator ports thereby preventing or limiting undesired bone anchor disassociation or backout.

The blocking cam and/or orthopedic fixation device can comprise positional lock features that determine a locked and unlocked mode such as complementing detents and dimples or ramps on the surfaces of the blocking cam and/or orthopedic device. Likewise, a positional lock feature on the blocking cam and/or elsewhere on the orthopedic fixation device provides the user a tactile response to indicate advancement to a locked or an unlocked mode.

Intervertebral spacer bodies 150A can assume different size profiles and heights depending on the patient needs and can be utilized in the lumbar, thoracic, or cervical spine. A variety of surgical approaches can be used such as anterior, lateral, or posterior. The profile of the intervertebral spacer can change depending on the surgical approach.

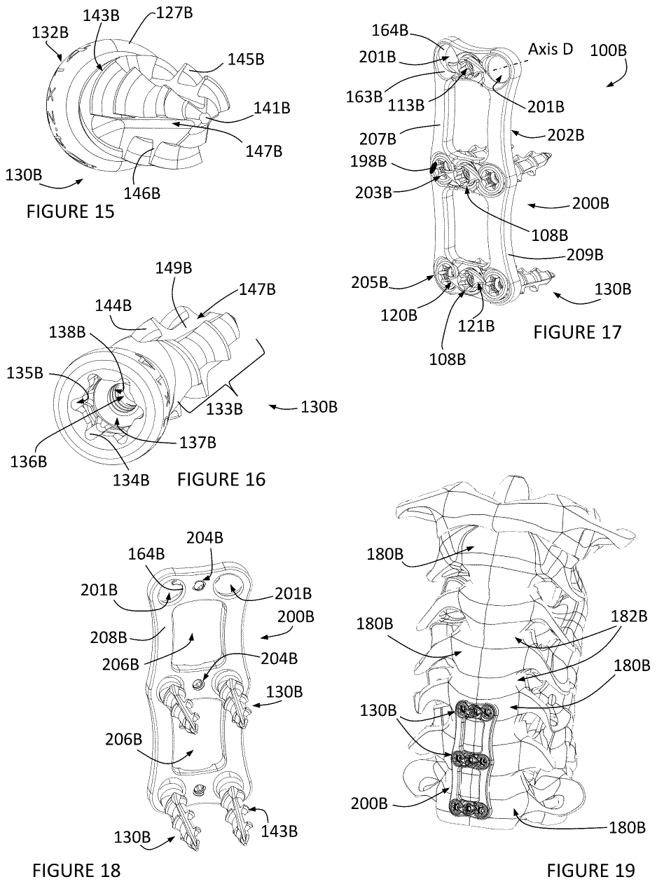

FIGS. 15-19 depict yet another embodiment of an implantable device utilizing an impacted bone anchor. In this embodiment, a bone plate is utilized in the stabilization of bone segments such as depicted in FIG. 19 where bone plate 200B is secured to the anterior aspects of adjacent vertebral bodies 180B that are separated by intervertebral discs 182B using a plurality of bone anchors 130B. In many respects, bone anchor 130B used here, is similar to bone anchor 130A depicted previously. Bone anchor 130B comprises an enlarged head 132B defined by a rounded head surface 127B with a screw body 133B extending from the enlarged head. Bone screw threads 143B extend along at least part of the length of the screw body. In this embodiment, bone anchor 130B has a plurality of axial grooves 147B that interrupt the bone screw threads 143B (helical flanges). The axial grooves are defined by rounded V-shaped groove face 149B extending from distal tip 141B proximally towards head surface 127B. Bone screw threads 143B have a distally facing distal thread face 145B and a proximally facing proximal thread face 144B with the threads reducing in diameter distally. Edge face 146B extends between distal thread face 145B and proximal thread face 144B and also merges with groove face 149B. Although the transition between the edge face and groove face is depicted as smooth, this transition can assume other profiles. For example, instead of a rounded V-shape, the axial grooves can assume other shapes such as U, have steps, or distinct intersections between the base and legs of the ‘V’.

In this embodiment of FIG. 15-19, bone anchor 130B has a similar head to bone anchor 130A. As noted in FIG. 16, inset into the head is head drive 137B for interfacing with the drive head of a driver tool, a head lock 136B having head threads 138B (for securing an inserter tool thereto) is distally placed from drive surfaces 134B that define drive recesses 135B. The drive recesses are utilized by an inserter tool 221B to drive the bone anchor into bone.

Bone plate 200B is in the form of a cervical plate, but of course can assume other plate configurations for other skeletal applications. The bone plate variations are well known in the art. As noted in FIG. 19, bone plate 200B spans across three levels of vertebral bodies 180B with intervertebral discs 182B therebetween. In alternative embodiments, the bone plate can be modified to span more or less levels by adding or subtracting fixator ports and adjusting its length accordingly. Bone plate 200B comprises a substantially flat plate body 202B which can be gently curved to follow the anatomy in which it is meant to cover. The bone facing side of the plate is defined by inner face 208B with an opposing outer face 207B facing away from the bone. Perimeter face 209B extends between the inner face and outer face and defines the outer boundary of the plate. Bone plate 200B comprises one or more first fixator ports 201B, one or more second fixator ports 203B spaced from the first fixator ports, and one or more third fixator ports 205B spaced even further from the first fixator ports along the length of the plate. The fixator ports extend through the outer face and inner face and comprise a tunnel face 163B that pass the enlarged head 132B and screw body 133B of the screw until narrowing at neck face 164B to a size that will not allow the enlarged head 132B to pass. One or more plate windows 206B can be present extending through the outer face and inner face.

Between or adjacent the fixator ports can be a receiver hole 204 for receiving a portion of a blocking cam 108B. Much like the blocking cams depicted in FIGS. 11-14, blocking cams 108B comprise a central drive pocket 113B configured to receive a camming tool 198B to rotate the blocking cam between a position wherein first tab 120B and second tab 121B interferes with the fixator ports thereby blocking bone anchors 130B from backout, to a position that doesn't interfere with the fixator port and thus provides for the insertion and removal of the bone anchors 130B.

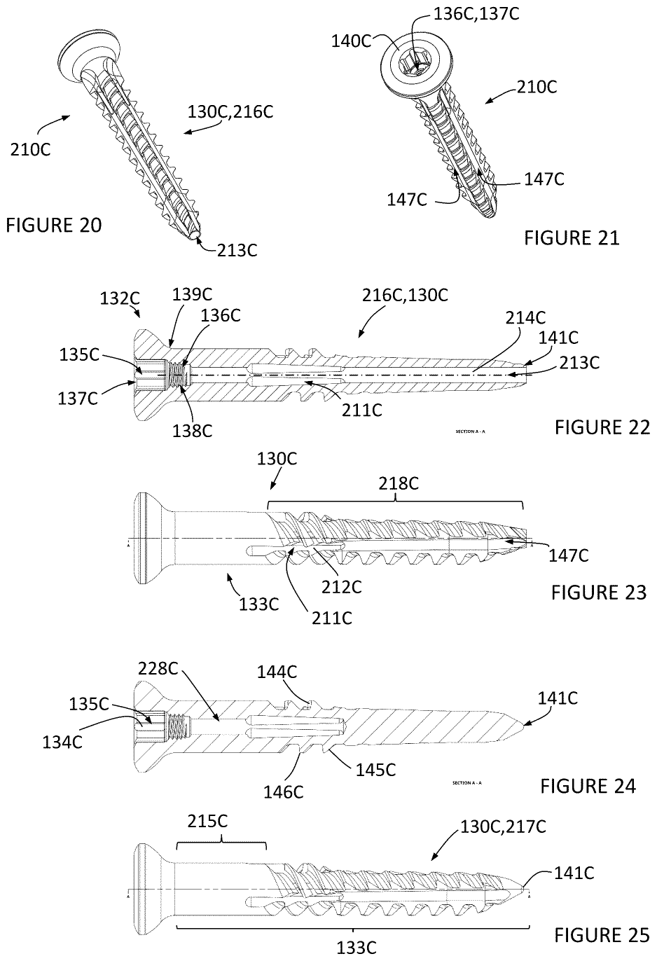

FIGS. 20-25 depict yet another embodiment of a bone anchor 130C which here is in the form of a sacral bone anchor 210C. This bone anchor again shares many of the same features as previously described bone anchors utilizing the same element numbers such as an enlarged head 132C, and a screw body 133C. In this embodiment, screw body 133C comprises a threaded/flanged portion 218C (having bone anchor threads/helical flanges) and an unthreaded/unflanged portion 215C. The drive recess 135C, head lock 136C, and head drive 137C, with head threads 138C are substantially the same as depicted for the anchor in FIG. 16. Note in this embodiment (FIG. 22-23), the bone anchor 130C can comprise a cannula 213C to form cannulated anchor 216C defined by a cylindrical cannula face 214C extending from distal tip 141C to proximal face 140C. Bone anchors depicted herein can comprise an elongate window face 212C defining a plurality of elongate windows extending through the bone screw. In some embodiments, the bone anchors are absent of a cannula such as those versions depicted in FIG. 24-25 and are termed solid anchors 217C. Despite the lack of a cannula extending the full length of the bone anchor, some embodiments can include an anchor inlet 228C which is a central bore that extends only part way into the bone anchor from the proximal end. The bone anchor depicted in FIG. 24-25 comprises an open drive recess 135C for receiving an anchor driver 221C, a head lock 136C which is a threaded cavity for receiving the threaded portion of the anchor driver, which then transitions to anchor inlet 228C defined by a cylindrical inlet surface, and ultimately to elongate windows 211C. It is through these passageways that bone material can be introduced into a bone anchor to enhance fusion across a joint.

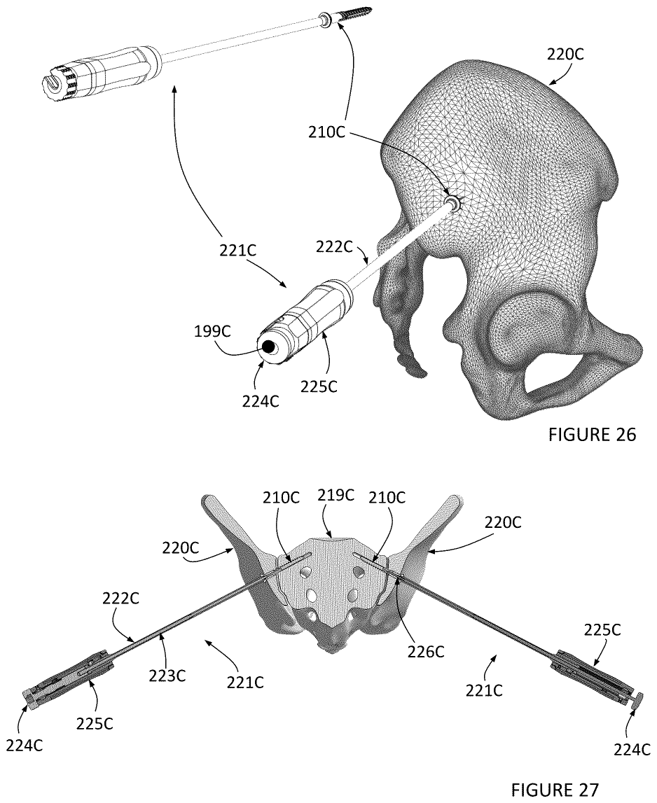

FIGS. 26-27 depict the use of sacral bone anchor 210C for fixation across a human sacral iliac joint. An anchor driver 221C is utilized to drive the bone anchor and comprises an elongate driver handle 225C extending from an outer drive shaft 222C in the form of a tube. Seated within the outer drive shaft is an inner drive shaft 223C that terminates in a threaded tip 226C configured for threading into head lock 136C of sacral bone anchor 210C. A capture knob 224C is accessible at a proximal end of the anchor driver such that the threaded tip 226C can be advanced into the threads of the sacral bone anchor. As noted in the Figures, sacral bone anchor 210C is driven, using an impactor tool 199C such as a surgical hammer, through each ilium 220C, across the sacral iliac joint, and into the sacrum 219C. When elongate window 211C is provided in the bone anchor, the elongate window is positioned such that it spans across the sacral iliac joint. Therefore, upon removal of inner driver shaft 223C, bone material or bone substitutes can be introduced through the cannula of the outer drive shaft 222C and pushed with a plunger down the cannula and through head drive 137C. Having the bone material pack the elongate windows enhances fusion across the sacral iliac joint by the bone material seeping out of the elongate windows 211C.

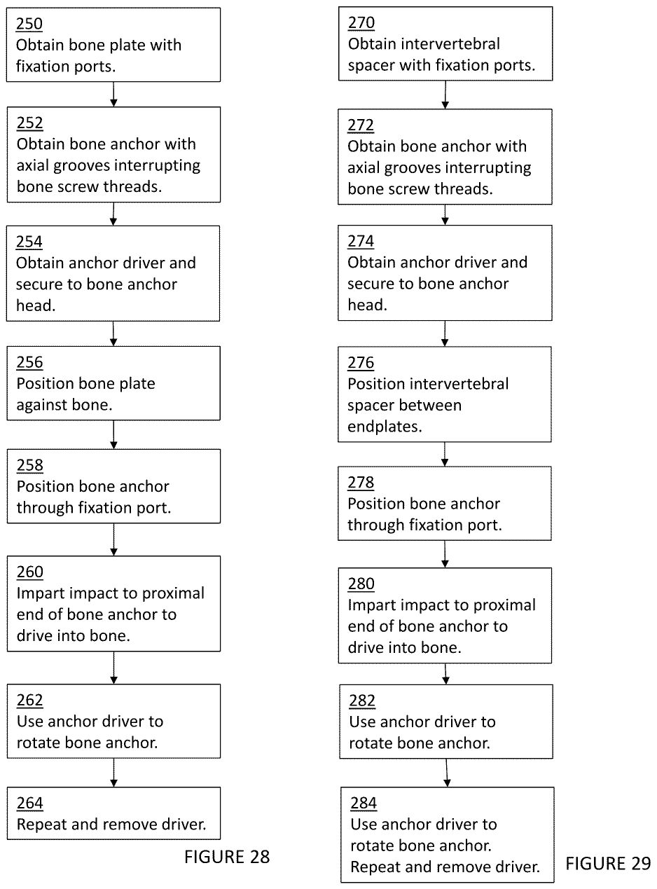

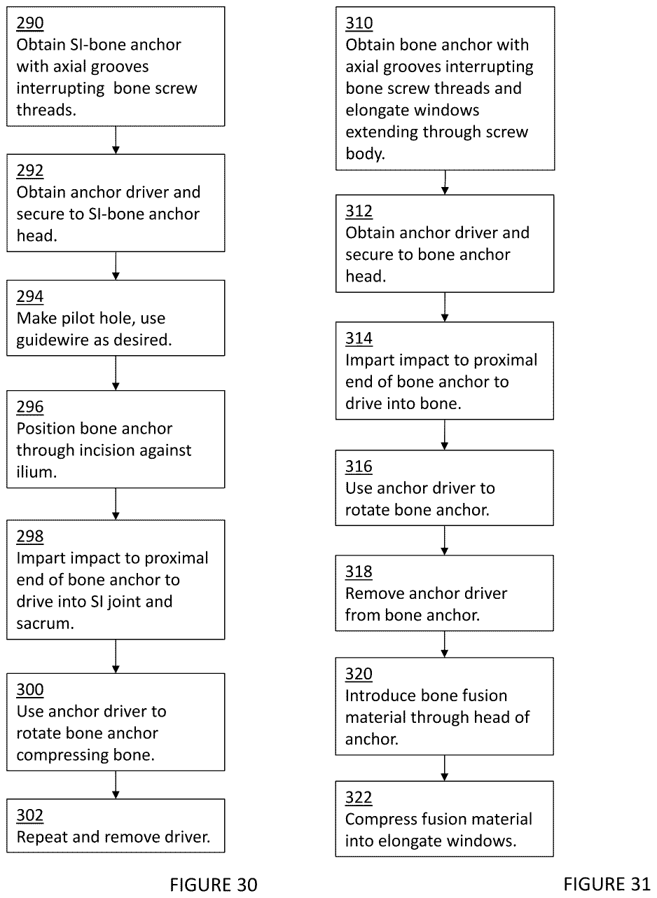

FIGS. 28-29 depict a method of using the disclosed bone anchors to facilitate a bone fusion by use of the bone anchor alone or in conjunction with an implant such as a bone plate, intervertebral spacer, a spinal rod, or motion preserving device such as an artificial disc. In securing a bone plate, the method begins with obtaining a bone plate with fixation ports extending through the bone plate (250). Obtaining bone anchors as disclosed herein, having axial grooves interrupting the bone screw threads (252). Obtaining a bone anchor driver and securing it to the head of the bone anchor (254). Positioning the bone plate against bone to be fixed (256). Positioning the distal tip of the bone anchor through a fixation port of the bone plate against the bone (258). Impacting the proximal end of the bone anchor to drive the bone anchor into bone (260). This impact can be via impact to an insertion instrument or directly to the head of the bone anchor. Applying rotational force on the anchor driver to rotate the bone anchor resulting in consequent compression of the bone (262). Repeating these steps for other bone anchors used to secure the bone plate and removing the driver from the bone anchor when complete (264).

In securing an intervertebral spacer (FIG. 29), the method begins with obtaining an intervertebral spacer with fixation ports extending through the spacer (270). Obtaining bone anchors as disclosed herein, having axial grooves interrupting the bone screw threads (272). Obtaining a bone anchor driver and securing it to the head of the bone anchor (274). Positioning the intervertebral spacer between endplates of adjacent vertebral bodies (276). Positioning the distal tip of the bone anchor through a fixation port of the intervertebral spacer (278). Impacting the proximal end of the bone anchor to drive the bone anchor into bone (280). This impact can be via impact to an insertion instrument or directly to the head of the bone anchor. Applying rotational force on the anchor driver to rotate the bone anchor resulting in consequent compression of the bone and securing the spacer in position (282). Repeating these steps for other bone anchors used to secure the intervertebral spacer and removing the driver from the bone anchor when complete (284).

When using the disclosed bone anchors to secure bone such as performed to stabilize a sacral-iliac (SI) joint, this can be done without use of other implant hardware. The method comprises obtaining a bone anchor (i.e. SI-bone anchor) having axial grooves (as described herein) interrupting the bone screw threads on the anchor (290). Obtaining an anchor driver and securing it to the anchor head (292). If so desired, making a pilot hole into bone and/or using a guidewire to guide a cannulated form of bone anchor into a predetermined position on the bone (294). Positioning the bone anchor through an incision against the ilium (296). Impacting the proximal end of the bone anchor to drive the bone anchor into bone (298). In this example, the impacted bone is the SI joint. This impact can be via impact to an insertion instrument or directly to the head of the bone anchor. Applying rotational force on the anchor driver to rotate the bone anchor resulting in consequent compression of the bone securing the spacer in position (300). Repeating these steps for other bone anchors used to secure the SI joint and removing the driver from the bone anchor when complete (302).

In some cases, the bone anchor(s) used will have a plurality of elongate windows extending through the screw body of the anchor. Note that the steps (310) through (318) replicate steps introduced previously to implant a bone anchor. However, note in step (310), the bone anchor also comprises elongate windows. Once the anchor driver is removed from the bone anchor (318), bone fusion material can then be introduced through the head of the anchor (320) and compressed to seep out of the elongate windows (322) to induce a fusion in the joint space.

Elements of the orthopedic fixation device can be constructed from a variety of implantable materials including polymers such as PEEK, metals such as Ti, and alloys such as Ti-64 and stainless steels. Many of these materials are considered non-porous or low porous. Additive manufacturing can be utilized where needed to create complex geometries. The intervertebral spacer can consist of a solid material, or as an open lattice structure.

Anatomical terms such as anterior and posterior are used with relation to placement of the intervertebral implant in a human body in an implanted mode.

It is noted that the terms “substantially” and “about” and “generally” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The foregoing invention has been described in accordance with the relevant legal standards, thus the description is exemplary rather than limiting in nature. Variations and modifications to the disclosed embodiment may become apparent to those skilled in the art and fall within the scope of the invention.

1) A bone anchor comprising:
a central anchor axis;
an enlarged head;
said enlarged head having a head drive for interfacing with a drive tool;
a distal tip at the distal end of the anchor;
a screw body;
said screw body extending between said distal tip and said enlarged head;
said screw body comprising a plurality of spaced helical flanges;
a plurality of axial grooves;
said axial grooves extending between said helical flanges between said distal tip and said enlarged head; and,
wherein said bone anchor is linearly impacted into bone during insertion.

2) The bone anchor of claim 1 further comprising:
a proximal face;
said proximal face on the proximal end of said bone anchor and facing proximally;
a cannula;
said cannula extending through said bone anchor between said distal tip and said proximal face and operable for receiving a guide wire.

3) The bone anchor of claim 1 further comprising:
a proximal face;
said proximal face on the proximal end of said bone anchor and facing proximally; and,
wherein said bone anchor is non-cannulated and therefore absent of a cannula extending from said distal tip to said proximal face.

4) The bone anchor of claim 1 further comprising:
said helical flanges comprising a distal thread face;
said helical flanges comprising a proximal thread face; and,
wherein said distal thread face is angled away from said distal tip.

5) The bone anchor of claim 1 further comprising:
said helical flanges comprising a distal thread face;
said helical flanges comprising a proximal thread face; and,
wherein said proximal thread face is angled substantially perpendicular to said central anchor axis.

6) The bone anchor of claim 1 further comprising:
a counter rotation body;
wherein said counter rotation body resists bone anchor rotation in a direction opposite insertion torque.

7) The bone anchor of claim 6 wherein said counter rotation body is positioned at the intersection of said helical flanges and said axial groove.

8) The bone anchor of claim 6 wherein said counter rotation body is in the form of one of a step and a barb.

9) The bone anchor of claim 1 further comprising:
an intervertebral spacer;
said intervertebral spacer comprising a fixator port extending through said intervertebral spacer; and wherein,
said bone anchor is driven linearly through said fixator port into bone to secure said intervertebral spacer between adjacent vertebral bodies.

10. The bone anchor of claim 1 further comprising:
a bone plate;
said bone plate comprising a fixator port extending through said bone plate; and,
wherein said bone anchor is driven linearly through said fixator port into bone to secure said bone plate against bone segments.

11. The bone anchor of claim 1 further comprising:
an anchor inlet extending at least partially into said screw body from a proximal end of said bone anchor along said central axis;
a first elongate window extending into said screw body ;
a second elongate window extending into said screw body;
wherein first said elongate window and said second elongate window intersect said anchor inlet within said screw body to provide fusion therebetween.

12. The bone anchor of claim 1 further comprising:
a non-threaded portion of said screw body;
said non-threaded portion adjacent said enlarged head.

13. The bone anchor of claim 1 wherein said screw body comprises helical flanges having a minor diameter that reduces in size as said helical flanges approach the distal end of said bone anchor.

14. The bone anchor of claim 1 wherein at least one of said helical flanges are misaligned across at least one of said axial grooves.

15. The bone anchor of claim 1 wherein said enlarged head is partially conical.

16. The bone anchor of claim 1 wherein said axial grooves converge distally towards said central anchor axis.

17. The bone anchor of claim 1 wherein said axial grooves are spaced parallel to said central anchor axis.

18. The bone anchor of claim 1 further comprising:
a head lock;
said head lock comprising head threads;
wherein said head threads are operable for releasable locking to an inserter instrument.

19. The bone anchor of claim 1 wherein said axial groove extends below the minor diameter of the bone anchor as defined by said helical flanges.

20. A method of fixating an orthopedic implant using a bone anchor comprising the steps of:
obtaining an orthopedic implant having a plurality of fixation ports;
obtaining a bone anchor comprising helical flanges and a plurality of axial grooves extending partially through said helical flanges between said enlarged head and said distal tip;
obtaining an anchor driver and attaching the anchor driver to the bone anchor head;
placing the distal tip of said bone anchor through said fixation port;
impacting a proximal end of said bone anchor driving the bone anchor into bone; and,
rotating the bone anchor to compress the bone.

21. The method of claim 20 further comprising the step of:
introducing bone material through the proximal end of the bone anchor for distribution through elongate windows in the bone anchor screw body.

22. A method of fixating a sacral iliac joint using a bone anchor comprising the steps of:
obtaining a bone anchor comprising a plurality of spaced helical flanges and a plurality of axial grooves extending partially through said spaced helical flanges;
obtaining an anchor driver and attaching the anchor driver to the bone anchor head;
placing the distal tip of said bone anchor against an ilium bone;
impacting a proximal end of said bone anchor to drive the bone anchor through the ilium bone into the adjacent sacrum; and,
rotating the bone anchor about its central axis.

23. The method of claim 22 further comprising the step of:
introducing bone material through the proximal end of the bone anchor for distribution through elongate windows in the bone anchor screw body.
